(19) United States
(12) Patent Application Publication
(10) Pub. No.: US 2015/0045631 A1
(43) Pub. Date: Feb. 12, 2015

(54) Skin health system

(71) Applicant: Lee Pederson, (US)

(72) Inventor: John Ademola, San Francisco, CA (US)

(21) Appl. No.: 13/999,709

(22) Filed: Mar. 15, 2014

Related U.S. Application Data
(60) Provisional application No. 61/852,394, filed on Mar. 15, 2013.

(51) Int. Cl. A61B 5/00; A61B 5/145; A61B 5/1455; A61B 5/0205

(52) U.S. Cl. CPC A61B 5/443; A61B 5/02055; A61B 5/14539; A61B 5/14551; A61B 5/0008; A61B 5/0022; A61B 5/681; A61B 5/486; A61B 5/02416
USPC 600/301

(57) ABSTRACT

An integrated system for skin and hair care provides customized personal analysis of a user's specific situation and supplies consumer product recommendations to the user. The system is implemented to allow a user to access their personal account via a mobile device. In another aspect the invention is a device, system and method for monitoring measurable skin qualities in real time to improve skin health, monitor specific medical conditions, and monitor the general health of an individual. The device is a wearable instrument package that measures and transmits skin and environmental measurement data. The system incorporates one or more such devices with a communication network and a remote data processor to improve the skin health and general health of an individual. The invention includes methods for monitoring skin health, general health, and specific medical conditions.

### FIELD OF THE INVENTION

The present invention relates to skin care, hair care and health care. More particularly, the present invention relates to a general skin health system, system components and methods for skin care and health care. The invention also relates to monitoring the state of health of an individual in real-time. In this aspect the present invention relates to a real-time skin monitoring system, system components and methods for monitoring general health using skin measurements in real-time. The invention also relates to wearable devices that monitor skin properties.

### PRIORITY CLAIM

This application claims priority to U.S. provisional patent application 61/852,394 for “Skin Health System” filed on Mar. 15, 2013.

### BACKGROUND OF THE INVENTION

Skin is the largest organ of the human body, and the most easily accessible to non-invasive monitoring of the health of an individual. In the context of a person's environment, measurements taken from skin can be correlated to a variety of physical conditionals. Additionally, skin health can be maintained and optimized by monitoring measurable skin qualities. At the same time, advances in instrumentation, communications, data processing, and personal medicine are revolutionizing human health care and the delivery of health services. There is a need for utilizing current and emerging technologies to optimize skin measurement and monitoring capabilities to improve both skin health and the overall health of individuals.

For consumers making purchasing decisions related to skin health, there is a need for mobile diagnosis, analysis and advice. Such services should entail technical comfort, social comfort, individuality, privacy and trust. The services should take into account the user's skin conditions, cultural and physical environment, lifestyle, and many other crucial factors. There is a need to integrate skin measurements devices, purchasing experiences, customer management, product/treatment systems, product lines, mobile telecommunications, medical technology and other relevant parameters for skin care and health care.

### BRIEF DESCRIPTION OF THE INVENTION

An integrated system for skin and hair care provides customized personal analysis of a user's specific situation and supplies consumer product recommendations to the user. The system is implemented to allow a user to access their personal account via a mobile device. In another aspect the invention is a device, system and method for monitoring measurable skin qualities in real time to improve skin health, monitor specific medical conditions, and monitor the general health of an individual. The device is a wearable instrument package that measures and transmits skin and environmental measurement data. The system incorporates one or more such devices with a communication network and a remote data processor to improve the skin health and general health of an individual. The invention includes methods for monitoring skin health, general health, and specific medical conditions.

### BRIEF DESCRIPTION OF THE DRAWINGS

The accompanying drawings, which are incorporated into and constitute a part of this specification, illustrate one or more embodiments of the present invention and, together with the detailed description, serve to explain the principles and implementations of the invention.

In the drawings:

FIG. 1 depicts an embodiment of the architecture of the skin health system.

FIG. 2 depicts a presently preferred embodiment of the architecture of the capabilities of the central hub.

FIG. 3 depicts a presently preferred embodiment of a service center component.

FIG. 4 depicts a presently preferred embodiment of a medical center component.

FIG. 5 depicts a presently preferred embodiment of a mobile device for use in the system.

FIG. 6 is a depiction of a presently preferred embodiment of the skin care app.

FIG. 7 is a depiction of a presently preferred embodiment of a skin care product supplier.

FIG. 8 is a depiction of a presently preferred embodiment of a service provider.

FIG. 9 is a depiction of a presently preferred embodiment of a research center.

FIG. 10 depicts a wearable device for noninvasive measurement of skin parameters.

FIG. 11 depicts a system for taking skin measurement data and communicating and processing the data.

### DETAILED DESCRIPTION

Embodiments of the present invention are described herein in the context of systems, devices and methods for monitoring and improving skin health and general health by measuring skin and environmental parameters by using noninvasive measurements. Those of ordinary skill in the art will realize that the following detailed description of the present invention is illustrative only and is not intended to be in any way limiting. Other embodiments of the present invention will readily suggest themselves to such skilled persons having the benefit of this disclosure. Reference will now be made in detail to implementations of the present invention as illustrated in the accompanying drawings. The same reference indicators will be used throughout the drawings and the following detailed description to refer to the same or like parts.

In the interest of clarity, not all of the routine features of the implementations described herein are shown and described. It will, of course, be appreciated that in the development of any such actual implementation, numerous implementation-specific decisions must be made in order to achieve the developer's specific goals, such as compliance with application- and business-related constraints, and that these specific goals will vary from one implementation to another and from one developer to another. Moreover, it will be appreciated that such a development effort might be complex and time-consuming, but would nevertheless be a routine undertaking of engineering for those of ordinary skill in the art having the benefit of this disclosure.

The skin should not be considered as an isolated organ but rather as a definite functioning system that communicates with the internal environment. Skin signs of systemic diseases occur frequently and sometimes feature the first symptoms of an internal disease; furthermore, these manifestations may be the sole expressions of otherwise asymptomatic systemic disorders. A number of dermatologic signs, symptoms, and disorders can be invaluable as markers of systemic disease. Although a plethora of specialized modern diagnostic tests are available, the skin still remains the only organ of the body that is immediately and completely accessible to direct clinical examination. The present invention monitors the skin signs of systemic diseases. The description of the clinical features of skin lesions and other skin properties observed in several internal diseases will be useful to general physicians, internists, and dermatologists in the diagnosis of a systemic disease.

In one aspect the invention is a mobile integrated skin health management system comprising: (1) a skin and/or hair analysis component, (2) a customer management system; (3) a product/treatment intelligent recommendation or treatment system; and (4) a complete testing and results database for consumers, home use, industries, clinical testing facilities, product manufacturers, professional facilities, medical facilities, hospitals, clinics and surgical facilities. The invention further includes individual components of the system, as well as methods related to the system and its components. The system is largely mobile telephone-based, employing existing devices and technologies currently owned and used by many consumers. The system enables skin diagnostics, skin testing, skin analysis, skin treatments and advice related to skin care and skin health. In one aspect, the invention may be specifically targeted to female users. In another aspect, the invention may be primarily targeted to cosmetics and beauty. In another aspect, the invention is applicable to hair. In yet another aspect, the invention involves monitoring, diagnosis, prevention and treatment of skin problems and/or diseases. The invention enables users to select or customize appropriate skin or hair care treatments or products for their own needs and occasions. The invention is intended to address many needs including cosmetic product selection, expert skin consultation or advice and accurate skin diagnosis and measurements.

The invention is also applicable to monitoring the general health of a user, or a particular health concern of a user. Non-invasive skin tests and measurements may be used as a mirror of the body as a whole to detect, prevent and mitigate medical problems. Additionally, such skin measurements may be used in conjunction with other data to increase the likelihood of maintaining or improving the general health of an individual. Other data may include genetic profiling, ethnicity, skin microbiology profiling, internal microbiology profiling, pregnancy status, ovulation status, non-invasive eye measurements, and other data related to an individual's physiology. Non-invasive eye measurement instruments may be incorporated into both the general skin health system and the real-time health monitoring system.

The invention enables users to select customized and appropriate skin care, hair care, hair color, and personal care products and/or services based on the conditions of their skin or hair, skin type, ethnicity, culture, moods, psychology, life style, climate, season, environment, immediate skin conditions and other factors.

One component of the system employs mobile telephones and/or other mobile devices for skin measurements, imaging, analysis, monitoring, product/treatments and accessing a consumer/customer management subsystem.

Another component of the system (the “service center” component) may be installed in multiple locations including kiosks, stores, shopping malls, beauty facilities, spas, image counters, hotels, golf courses, student centers, gyms, airports, clubs, resorts, mobile facilities and the like for personalized service by trained technicians.

Another component of the system may be installed in multiple locations including dermatology clinics, medical spas, hospitals, medical laboratories, academic institutions, mobile facilities and the like to be used by medical professionals for skin diagnosis and skin treatments or skin care recommendation for patients (the “medical center” component). The system can also be installed in clinical testing labs for proof of claims substantiation studies.

Yet another component of the system may be installed at multiple locations including research centers, product testing centers, mobile facilities and the like to be used by product developers and/or product testers (the “research center” component).

The invention may be routinely accessed by a user at home, at work, or while traveling whenever a user wants to check their skin status/condition or desires advice on products and/or services.

The system is meant to be useful over the entire lifetime of a user. Personal histories and medical data may be stored indefinitely. Since skin is a mirror to the entire body's health, the information stored in the system may be applicable to other aspects of a user's personal health. The system is designed to respond to aging, changes of the seasons, and as a user moves between geographic locations. For example, the app may detect a change in the user's location and recommend an appropriate specific product or treatment, e.g. a product for extra moisturizing given the skin type and skin condition of the user.

The system may include a feature that creates a 3D high-definition image of a user's face, skin or other body parts. The system can then model the likely effects of aging and or treatments on conditions such as wrinkles, acne, stretch marks, skin pores, skin tone, pigmentation, and the like.

A user may input information concerning personal cosmetic needs in treating the face, skin, or body feature while also capturing a current image of the face, skin or body feature. The system will model the information provided to reflect the likely effect of a programmed, preselected, input or recommended cosmetic treatment over a period of time (e.g. skin tone, wrinkle, sags, textures, radiance or other skin parameters). Images of the likely transformation for different treatments may be compared until the user chooses an optimal transformation that makes them happy.

The system is implemented so that the location of the individual user and other people supporting the system is flexible. For example, the individual user typically need not be in the same location as a medical professional providing a diagnosis. Similarly, technicians associated with the central hub need not be in any specific location, and even the personnel and equipment of the central hub may be dispersed in various locations.

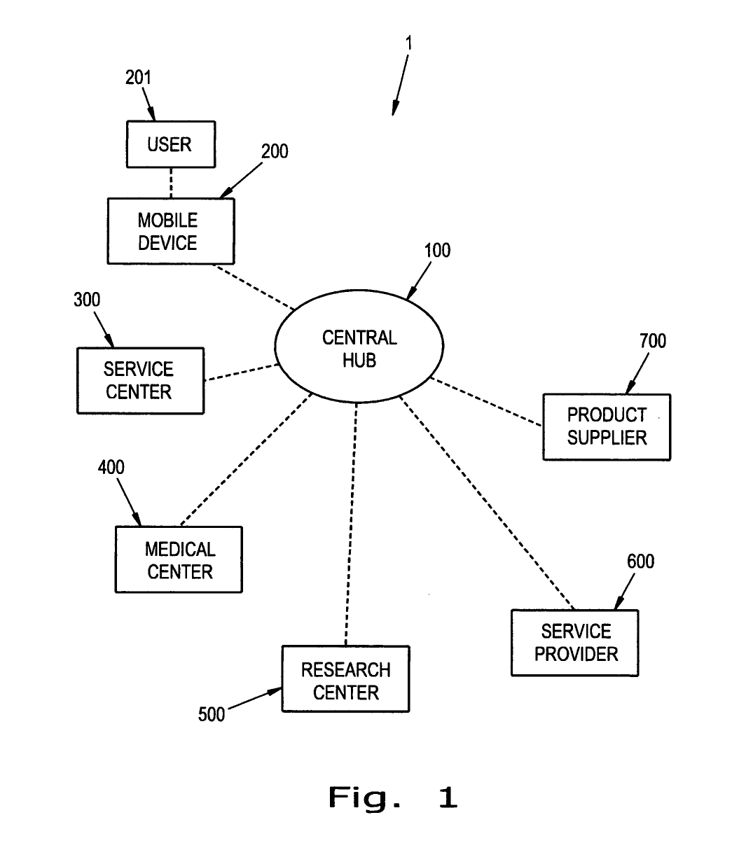

FIG. 1 depicts a presently preferred embodiment of the architecture of the skin health system for testing and treating skin. The system 1 includes central hub 100. Hub 100 serves as an interface between the data and analytical routines and the system users. Hub 100 serves as a connector and communications facilitator between users and components of the system. Hub 100 also includes human resources which provide human attention and assistance to system users. The users may be individual users, service centers and their agents, medical centers and their agents, or research centers and their agents. The system further may include a plurality of mobile devices 200 with installed apps, services centers 300, medical centers 400, research centers 500, service providers 600, and/or product providers 700. In some cases certain locations or persons may have more than one function in the system. For example, a service center may also serve as a service provider. The system may be implemented without including all the components described herein, though the central hub 100 and mobile device 200 with app are essential. An individual user 201 employs the mobile device 200 with installed app to interact with the system 1.

Alternative architectures of the system may be implemented, and all communications between users and components need not pass through the central hub.

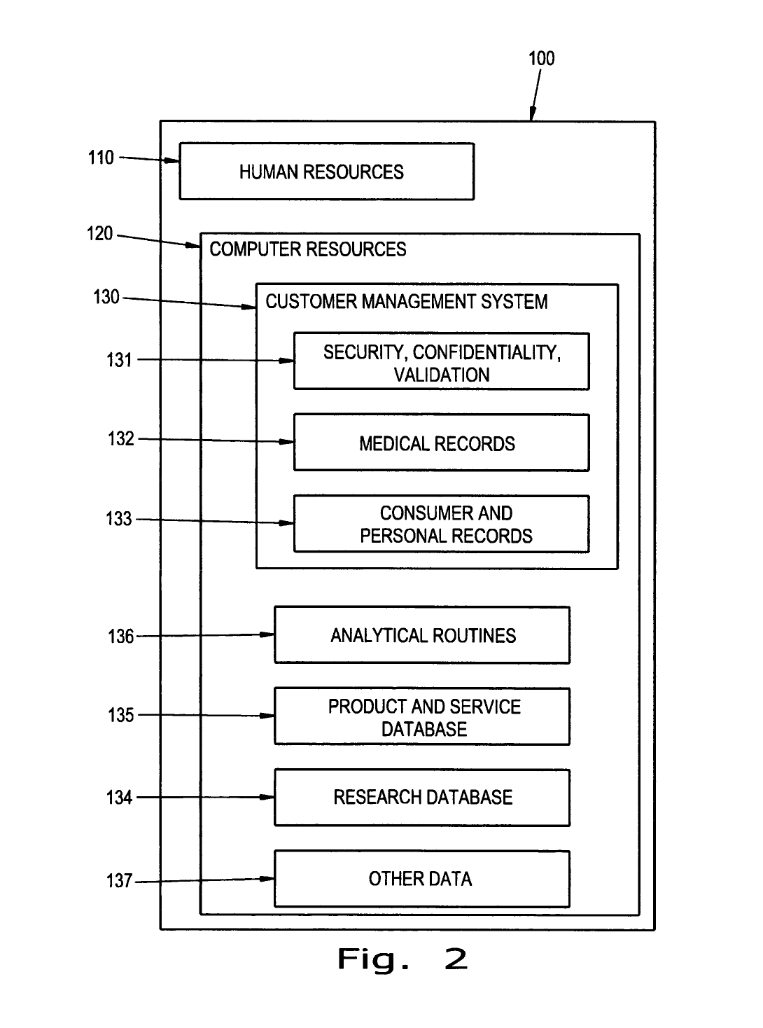

FIG. 2 depicts a presently preferred embodiment of the architecture of the capabilities of the central hub 100. Hub 100 has two main components, human resources 110 and computer resources 120. Human resources 110 includes live persons who may provide personal assistance to user 201 (see FIG. 1). Computer resources 120 includes hardware and software such as servers, data storage units, databases, and analytical routines. Customer management system 130 provides capabilities for establishing and maintaining personal records for each individual user 201. Security function 131 provides security, confidentiality and validation for the personal data of each user 201. Security function 131 allows or prevents access to personal records depending on authorization. Access may be segmented according to the status of the system component requesting access. For example, medical centers 400 (FIG. 1) may have access to different aspects of personal records than a service center 300 (FIG. 1). Medical records 132 comprises medical data, including health histories, testing and results. Consumer and personal data 133 comprises data relating to a user's consumer preferences, recommendations and history as well as health status data collected by non-medical professionals, e.g., pictures of a user's skin taken by the user. Another function of computer resources 120 is research databases 134. Research databases 134 includes general medical and research information that may be accessed by other components of the system. Another function is product databases 135, which comprises detailed data on individual skin care products including compatibilities and likely outcomes when used by user's having specific individual profiles, i.e., medical history, allergies, ethnicity, personal preferences, etc. Analytical routines function 136 includes software and algorithms to access appropriate data and inputs and then produce suggestions, warnings, data, informational material, and the like to various components of the system. Analytical routine function 136 uses the information in the databases as well as other information to provide treatment options and forecast likely outcomes for specific treatments for specific users. Other data 137 is also stored in the databases.

For each individual user 201, databases 132, 133 store data on skin measurements, skin diagnoses, images, and analyses. Such data may include data on texture (pores), sags/lines/wrinkles, radiance/color, skin physiology, skin biochemistry, skin condition, environment, culture, mood, psychology, personality, skin care history, skin product history, skin care preferences, skin sensitivity, skin allergy, genetic history, ethnicity and the like. New parameters may be added as technology, research, science, medicine and user personality, age, environment, health conditions, etc changes. Current diagnoses and other parameters can be consulted and compared with previous measurements.

Many physiological measurements related to a user's skin health and general health may be stored in the databases. These measurements may be obtained at service centers 300, medical centers 400, research centers 500, or by a user 201 using a measurement instrument connected or incorporated into to their mobile device 200. Such measurements include blood pressure, cholesterol (HDL and LDL), glucose levels, endocrine glands variation, apocrine glands variation, sebaceous gland variation, stratum corneum structure and water loss, pigmentation related tanability, skin vs diabetic conditions, skin vs lung functions, skin vs cholesterol level, skin vs mental alertness, skin vs brain functions and other health and body parameters.

Different locations of a user's body may be separately tested, so that body part-specific products and treatments may be recommended and employed.

Instruments for obtaining the measurements include cutometers, pulse oximeters, corneometers, Towameters, Veriscans, and the like, as well as laboratory equipment and tests (including “lab on a chip” technology) for chemical and biological analysis.

Measurements and diagnostics may be linked to treatment devices such as iontophoretic delivery systems, cosmetic acne patches, anti aging devices and the like. Communication within the system 1 may allow treatments to be delivered in a pulsatile or controlled manner with appropriate monitoring by professionals. Mobile devices may connect with one or more treatment device controllers for directing lasers, ultrasonic wands, iontophoresis wands, and the like. Both input and controller connections are implemented as high-speed data links such as USB, Firewire, Bluetooth or the like.

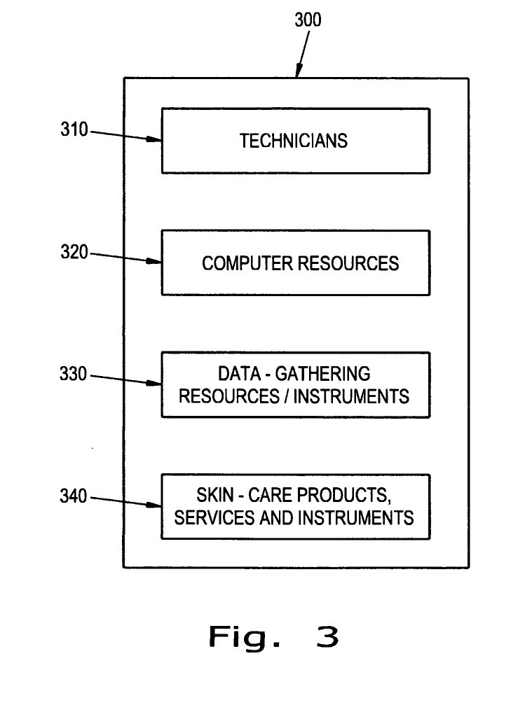

FIG. 3 depicts a presently preferred embodiment of the service center component. Skin care service center 300 is a physical location where an individual user 201 may access personalized skin care resources. Service center 300 includes one or more technicians 310, computer resources 320 that are used to connect with and interact with the system 1, and data-gathering resources 330 that are used to collect personal data and measurements of a user 201. Data gathering resources include chemical and physical measuring and analytical devices such as described elsewhere in this specification. Data gathering devices also includes data input devices, e.g. for inputting the personal history and preferences of a user/consumer. Service center 300 maintains an inventory of skin care products 340 that technician 310 may provide to user 201 based on guidance provided by the system 1. Service center 340 also provides facilities and services for use in treating a user 201 by a technician 310.

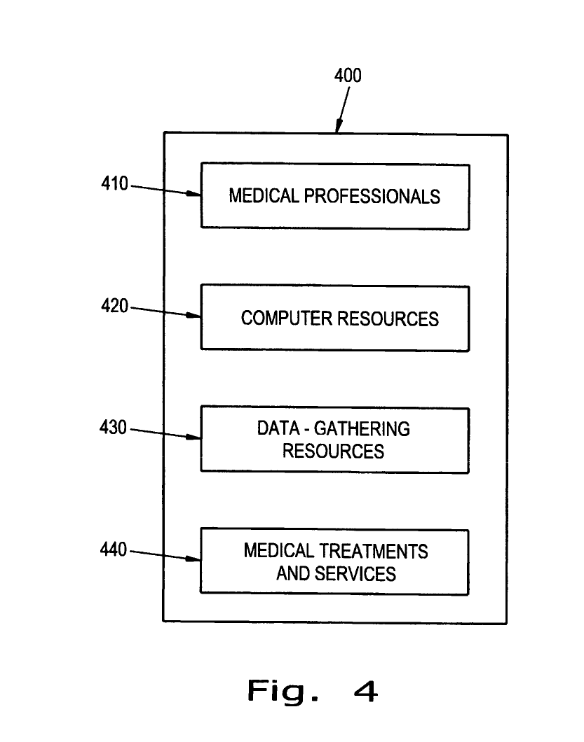

FIG. 4 depicts a presently preferred embodiment of the medical center component. Medical center 400 is a physical location where an individual user 201 may receive medical diagnosis and/or treatment. Medical center 400 includes one or more medical professionals 410, computer resources 420 that are used to connect with and interact with the system 1, and data-gathering resources 430 that are used to collect medical data and measurements of a user. Data gathering resources include chemical and physical measuring and analytical devices such as described elsewhere in this specification. Data gathering devices also includes data input devices, e.g. for inputting the medical history and diagnoses of medical professionals. Medical center 400 may provide prescriptions and medical services such as surgery as well as other treatments. Medical center 400 may also keep a database medical records of a user.

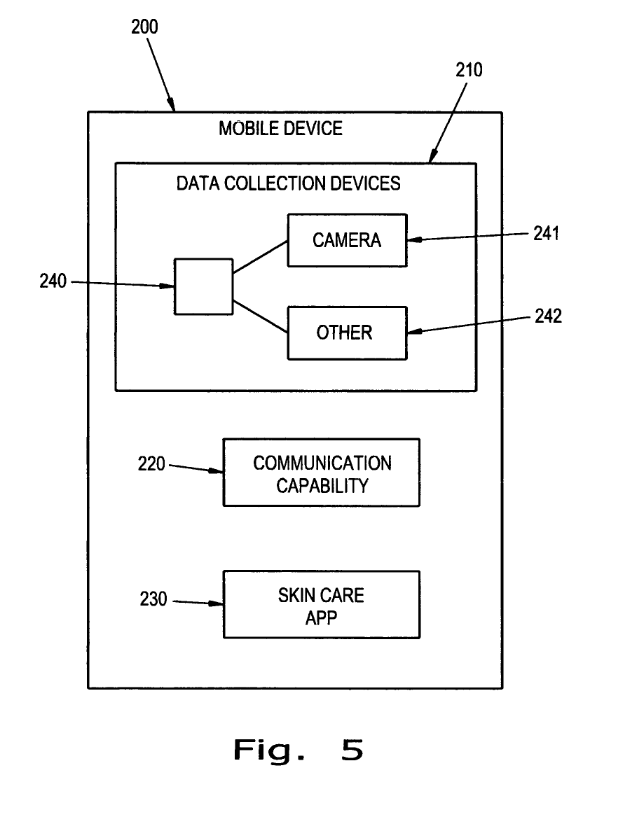

FIG. 5 depicts a presently preferred embodiment of a mobile device for use in the system. Mobile device 200 comprises a cell phone, laptop computer, tablet computer or the like. Mobile device 200 includes data collection devices 210 and/or connections to data collection devices 240. Such data collection devices may include a camera 241 and/or other devices such as described elsewhere in this specification. Mobile device 200 includes communication capability 220 which enables the devices to connect and interact with system 1. Importantly, mobile device includes skin care app 230.

One component of the invention is an app for mobile communication devices. An individual user 201 may download the app from the AppStore and other wireless telephony systems. The app may be implemented such that there is no financial commitment on the consumer's part and no payment to download the application. Versions of the app may be used on all carriers and cell phone models.

The app uses the system 1 to provide to the user advanced knowledge/diagnosis of an individual user's immediate and long term skin conditions so the user can always take better care of their skin or make better choice of skin care products and/or treatments for their skin personality.

Features of the app may include a combination of the following functions for an individual user: ability to order products directly and have them shipped to a desired destination; ability to rate and review products; ability to view customer ratings and reviews from others; ability to identify and locate nearby skin care technicians and professionals; and information on environmental, chemical or biological threats to skin, health, or person.

The app may be used as a security and personal recognition system since it may recognize unique skin and hair parameters. The app may link to a dermatologist providing diagnostic testing and results in few minutes. The app may be used to monitor and document skins conditions including acne, eczema, psoriasis, skin infections, age spots, moles, melanoma, precancerous conditions and the like. The app may provide information on skin care treatments, products (actions, results, advice, ingredients), safety data and claims. The app may provide information on skin care manufacturers and services providers, including ratings by other users. The app my be used to locate nearby products and services. The app may aid a skin care provider or advisor to provide customized services, products and advisory services to help the user take better care for their skin. The app may be used by product and service providers to advertise and present information to potential customers and clients. The app may be used by the user to rate products and services.

The app may include a loyalty card feature or discount feature for providers of products and/or service. The app may include a “latest news” feature that provide information on new research related to skin health.

The app may be specifically tailored to provide information and advice depending on the user's gender, age, ethnicity, lifestyle, geography or other factors that may make a difference in an individual user's specific needs, concerns and/or interests. For example, the app may suggest service providers that especially cater to the needs of women or men as the case may be, e.g. a skin salon that has a sports bar theme may have more demographic appeal to men than women. The app may include a feature that measures UV or other sun exposure in the user's location and recommends specific treatments or actions, e.g., SPF of sunblock, limitation on time in the sunlight, need for vitamin supplements (during periods of low sun exposure), and so on.

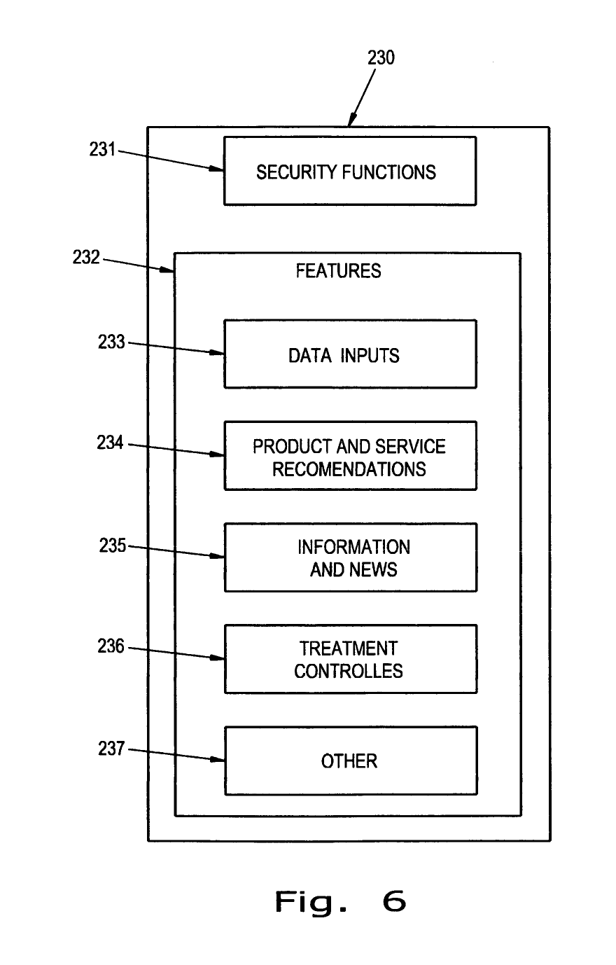

FIG. 6 is a depiction of a presently preferred embodiment of the skin care app. Skin care app 230 includes security mechanisms 231 that interact with customer management system 130 to protect a user's personal information and medical records. Such security mechanisms may include passwords and or biometric information obtained by a camera or other instrument. Skin care app 230 also has many functions 232, which may include data inputs 233 (both language-based and instrument-based), product and service recommendations 234, information and news 235, treatment controllers 236 and other functions as described elsewhere in this specification.

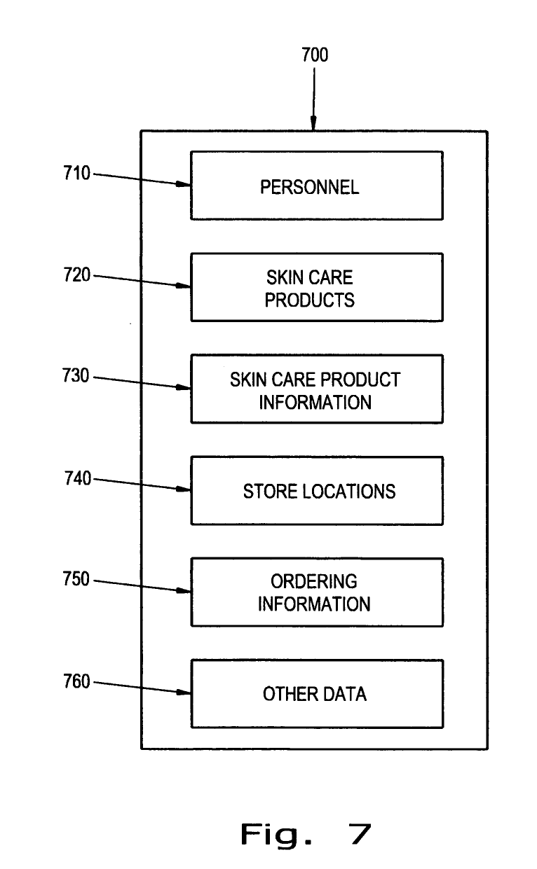

FIG. 7 is a depiction of a presently preferred embodiment of a skin care product supplier. Skin care product supplier 700 is an entity in the business of proving and selling skin care products. Product supplier 700 includes personnel 710 and sells one or more skin care products 720. Product supplier 700 interacts with the system by listing its products 720 in products and services database 135 (FIG. 2) and listing skin care product information 730 in research database 134 (FIG. 2). Store location data 740, ordering information 750 and other data 760 are also archived at computer resources 120 of central hub 100 (FIG. 2). User 1 may access all this data by mobile device 200. Further, analytical routines may suggest products 720 to user 1. A skin care product supplier 700 may be the same entity as a service center 300, a service provider 600 or other entity. Skin product supplier 700 may use the system 1 to target product advertisements to specific users via their mobile devices.

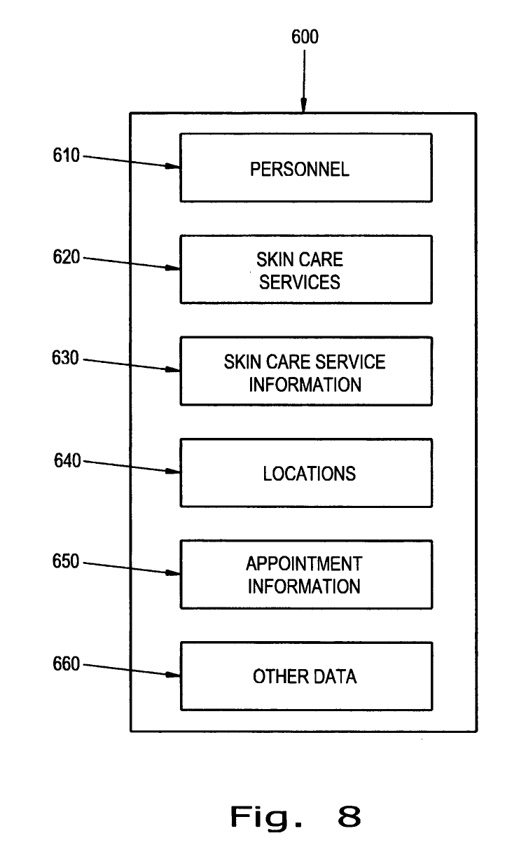

FIG. 8 is a depiction of a presently preferred embodiment of a service provider. Skin care service provider 600 is an entity in the business of proving and selling skin care services. Service provider 600 includes personnel 610 and sells one or more skin care services 620. Service provider 600 interacts with the system 1 by listing its services 620 in products and services database 135 (FIG. 2) and listing skin care service information 630 in research database 134 (FIG. 2). Location data 660, appointment information 650 and other data 660 are also archived at computer resources 120 of central hub 100 (FIG. 2). User 1 may access all this data by mobile device 200. Further, analytical routines may suggest services 620 to user 1. A skin care service provider 600 may be the same entity as a service center 300, a product supplier 700 or other entity. Service provider 600 may use the system 1 to target advertisements for services to specific users via their mobile devices.

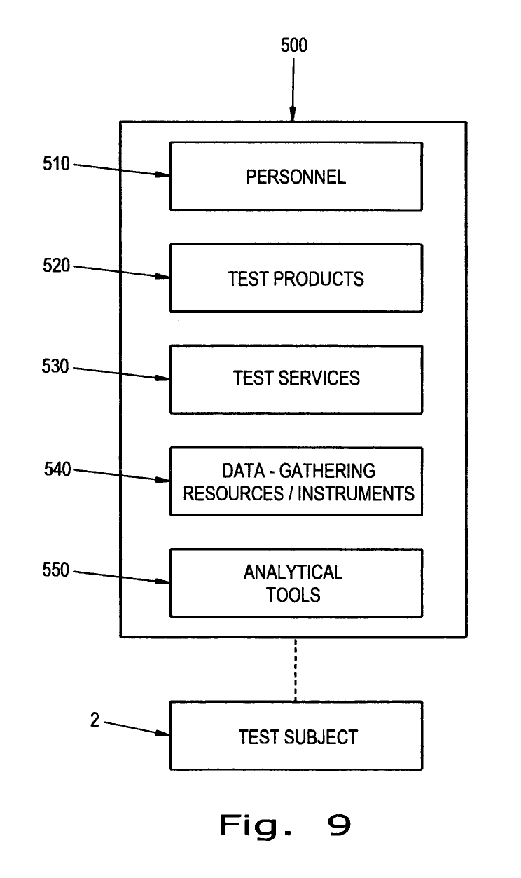

FIG. 9 is a depiction of a presently preferred embodiment of a research center. Research center 500 is an entity whose purpose is to test existing and potential products and services, and then collect and analyze the testing results. Research center 500 includes personnel 510, data-gathering resources/instruments 540 and analytical tools 550. Test products 520 and test services 530 are evaluated at research center 500 by personnel 510 using data-gathering resources/instruments 540 such as surveys, personal histories, and other measurement tools and measurements described elsewhere in this specification. Then the results are analyzed using analytical tools 550 including computer routines and algorithms designed to assess safety and effectiveness of products and services, often for specific environmental and personalized situations. The results of these evaluations may be archived at research databases 134 to be used by analytical routines 136 to suggest appropriate products, treatments and services to user 201. Test subjects 2 may be used in evaluating products and services.

In one aspect, the invention is an integrated skin care/skin health system that connects an individual user using a mobile device to skin care products and skin care services centers via a central hub. The system may comprise a communications channel to connect an individual user using a mobile device to the hub; and a central hub having computer resources including a customer management subsystem having security capabilities and storing consumer and personal data for the user, a products and services database, and analytical routines for using the consumer and personal data and the products and services database to suggest products and services to the user using a mobile device.

In another aspect the invention is a central hub for an integrated skin care/skin health system that may connect an individual user/consumer with product suppliers, service centers, health care providers, research centers, and service providers. The hub may comprise: human resources including persons trained in assisting a user with their personal skin care needs and operation of the system; and computer resources including a customer management subsystem having a security function to validate the identity of a user; a database storing personal information of the user related to skin care and skin health; a database storing information related to skin care products and/or services; and analytical routines using the information in the database to provide skin care product and/or service recommendations to the user.

In another aspect, the invention is an app for a mobile communication device. In this aspect the invention is an app for a mobile communication device. The app may comprise: a security function to verify the identity of a user; and a product and/or service recommendation function linking to remote databases storing information related to the user's skin care needs and preferences and information related to skin care products and/or services and using the information to provide recommendations of skin care products and/or services to the user. The app may further comprise a data input function for collecting data on the user's skin care needs and preferences. The data may include an image taken by a camera incorporated the mobile communication device. The security function may use an image taken by a camera incorporated the mobile communication device to verify the identity of the user. The app may further comprise an appointment function allowing the user to arrange an appointment function with a skin care service provider. The app may further comprise an ordering function allowing the user to order skin care products.

In another aspect, the invention is a method of monitoring skin health of a user. The method may comprise capturing new data related to the user's skin condition using a mobile communication device; transmitting the data to a central hub having a database storing existing data about the user's skin condition; and comparing the new data to the existing data using a computer routine to produce an analysis. The method may further comprise transmitting the analysis to the user via the mobile device. The new data may be a digital image. Conditions that may be monitored include moles, age spots (liver spots), eczema, psoriasis, acne, melanoma, and precancerous skin lesions (actinic keratosis).

The method may comprise the steps of: (a) collecting input information from the consumer's skin regarding a plurality of characteristics associated with skin condition of the consumer from home, retail centers, research laboratory, kiosks etc; (b) using the input information and measured value to identify skin care products for the consumer; and (c) providing the consumer with information regarding the skin condition, and the effects of the skin care treatment or product the consumer is applying to the skin. (d) providing a personal care regimen to the consumer thereby allowing the consumer to use the personal care regimen in a manner to provide optimum health for the skin of the consumer. (e) providing the consumer with information regarding skin conditions; and (f) recommending to the consumer products, treatments and advisors to improve skin and scalp health. Input measurements may include hydration, sebum, pigmentation, elasticity, pH, water loss, skin wrinkle parameters, aging parameters, skin tone and others. The system uses the telephone's existing camera for imaging the skin. Additional measuring devices may be connected to the phone or wireless device. The data can be used to identify consumers skin care needs, link to skin treatment or product database or bank to help consumer choose appropriate skin regimen, or to monitor the conditions of the skin, or help consumer to determine nutrients, foods, water, and other skin needs. The data can either be wirelessly transmitted to the user, or may be transmitted from skin care centers, retail locations, or medical centers. In addition to individual measurements, the App and software may provide the customers a report on conditions affecting the skin, such as local sun exposure, pollution, humidity and the like.

In another aspect the invention is a method of providing personalized skin care services. The method may comprise: storing personal information about a user in a database; storing information on the user's skin condition in a database; storing information of a plurality skin care services in database; allowing the user to request a recommendation for a skin care service via a mobile device; using the information in the database and an analytical routine to provide a recommendation; and sending the recommendation to the user via the mobile device.

In another aspect the invention is a method of collecting data for product safety, product effectiveness, and product development. The method may comprise: maintaining a skin care research facility; maintaining a central hub having human resources including persons trained in assisting a user with their personal skin care needs and operation of the system; and computer resources including a customer management subsystem having a security function to validate the identity of a user; a database storing personal information of the user related to skin care and skin health; a database storing information related to skin care products and/or services; testing skin care products and/or services on test subjects at the research center; and transferring the results of the testing to the central hub database.

In another aspect the invention is a method of advertising and promoting skin care products and/or services. The method may comprise: maintaining a central hub having computer resources including a customer management subsystem having a security function to validate the identity of a user; a database storing personal information of the user related to skin care and skin health of the user; a database storing information related to skin care products and/or services; receiving a request from a user for a recommendation for skin care products and/or services, where the request is sent to the central hub by the user using a mobile communication device; comparing the request and the information in the database to provide and appropriate recommendation; and transmitting the recommendation to the user via the user's mobile communication device.

In another aspect the invention is a method for allowing a user to arrange a skin care appointment. The method may comprise: storing personal information about a user in a database; storing information on the user's skin care condition in a database; storing information of a plurality skin care providers in database; allowing the user to request a recommendation for a skin care appointment via a mobile device; using the information in the database and an analytical routine to provide a recommendation; and sending the recommendation to the user via the mobile device.

In another aspect the invention is a method for allowing a user to select a skin care product. The method may comprise: storing personal information about a user in a database; storing information on the user's skin condition in a database; storing information of a plurality skin care products in database; allowing the user to request a recommendation for a skin care product via a mobile device; using the information in the database and an analytical routine to provide a recommendation; and sending the recommendation to the user via the mobile device.

Though this specification primarily uses the terms skin care, skin health, skin care product(s), skin care service(s) and the like, it is intended that the invention is equally applicable to hair care, hair health, hair care product(s), hair care service(s) and the like in the consumer (i.e., non-medical) realm.

In yet another aspect the invention is a device, system and method for monitoring measurable skin qualities to improve skin health, monitor and detect specific medical conditions, and monitor the general health of an individual. The device is a wearable instrument package that measures and transmits skin and environmental measurement data. The system incorporates one or more such devices with a communication network and a remote data processor to improve the skin health and general health of an individual. The invention includes method for monitoring skin health, general health, and specific medical conditions.

The system and device have a broad range of applications. They may be used to monitor the general health condition of an individual. They may be used to monitor specific medical conditions such as congestive heart failure and heat stroke, dermatological manifestations of non-infectious internal diseases, immune, autoimmune and rheumatic diseases; genetic, metabolic, endocrine and nutritional states, haematological diseases; psychiatric states; and drug eruptions.

They may be used as an aid in athletic training and physical rehabilitation. They may be used to monitor skin health and provide recommendations for improving skin health.

The wearable device may be configured in a variety of different ways depending on the particular applications. The three main variables in configuring the device are the design of the device for securing it against the desired skin area (e.g., wrist, upper arm, neck, torso, scalp, ankle, etc. . . . ); the complement of skin measuring instruments; and the complement of air measuring instruments. The invention contemplates and includes a wide range of configurations. Though the invention is not limited to the configurations described below, the following are provided as examples.

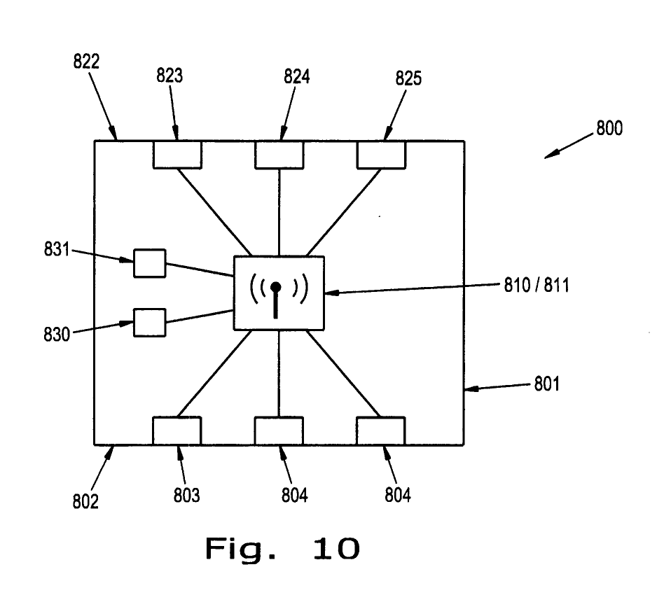

FIG. 10 depicts the functional components of a wearable device 800 for noninvasive measurement of skin parameters. Wearable device includes a housing 801 comprised of suitable materials such as fabric, plastic and metal. The housing 801 supports or is integrated with a skin contact area 802 having one or more skin measurement instruments 803, 804 and 805. The particular instruments in any one device will vary according to the specific application of the device 800. Instruments 803, 804 and 805 take skin measurements and send the data to transmitter 810 which in turn connects with a system outside the device 800. The device may also include an air contact area 822 having one or more skin measurement instruments 823, 824 and 825. Instruments 823, 824 and 825 may measure environmental factors such as air temperature, relative humidity, UV exposure and the like. This environmental measurement data is also send to transmitter 810. The device may also include a receiver 811 to receive information from the system. Device 800 may also include other components such as a GPS module 830 or alarm 831, which may notify the device wearer if critical limits such as UV exposure, blood pressure, or body temperature are met or exceeded.

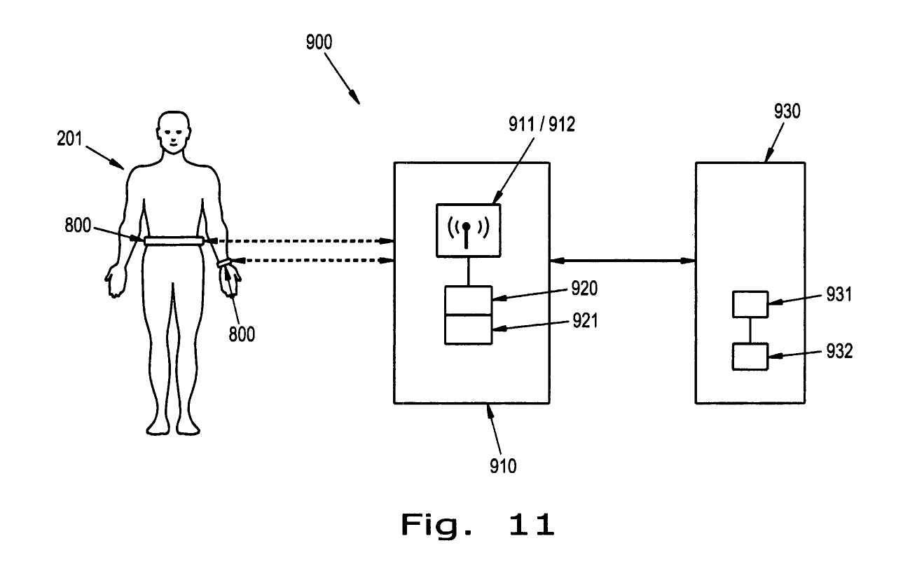

FIG. 11 depicts a system for taking skin measurement data and communicating and processing the data. Real-time skin monitoring system 900 includes wearable devices 800, which may be worn by user 201. Each device 800 communicates with local unit 910, which may be a cell phone or other communication device having transmitter 912 and receiver 912. Local unit may optionally include a database 920 and processor 921 for real-time monitoring. More preferably, local unit 910 communicates with central computing unit 930, which has database 931 and processor. Central computing unit 930 may be the same as the central hub 100 of skin health system 1. A plurality of wearable devices may be used at the same time, with each of the devices designed for particular areas of skin and designed to take particular measurements.

The real-time skin monitoring system has many applications and embodiments. In one embodiment, the system may be used for warning of a potential heat stroke, for example by football players undergoing conditioning exercises in hot weather. In this embodiment, the wearable device or devices may measure perspiration rate, heart rate, air temperature and relative humidity. The database may include information on the user's normal heart rate, body mass index, general level of conditioning and other relevant factors. The processor continually processes the incoming data, calculates internal body temperature of the user, and sends a signal (alarm) if the user's body temperature begins approaching an unsafe level.

In another embodiment, the real-time skin monitoring system may be used by a beach-goer to limit exposure to the sun. In this embodiment, the wearable device may monitor skin hydration and UV exposure and the database may contain information on previous UV exposure and the current level of tanning of the skin. The device may be incorporated into a swimsuit to prevent extra tan lines, or if it is worn on an exposed area of skin it may include a timer and alarm to remind the user to move it to a different area periodically. Other embodiments of the real-time skin monitoring system will be readily apparent to those having benefit of this disclosure.

Many systemic or internal diseases manifest in skin symptoms. Also, pharmaceutical drugs and their metabolites typically find their way to the skin. There may be skin signs of an otherwise nonobviousness internal malignancy or skin signs of an internal disease other than a malignancy. Further, internal malignancies and diseases may correlate with measurable skin properties. Often, skin symptoms are a valuable tool in diagnosing specific diseases. In this rapidly advancing area of research, it is likely that many or even most diseases (as well as the side effects of disease treatment) may someday be detectable by measuring skin properties and/or detecting specific chemical compounds in the skin. Further, as technology improves it is likely that early and preventive diagnoses may be obtained by combining skin measurements with a person's medical information including gene sequences and gene expression data.

Both the general skin health system depicted in FIG. 1 and the skin monitoring system shown in FIG. 11 may be implemented using current technologies. However, many additional implementations of the inventions described herein will be possible in the future as technical and medical knowledge advances. Presently unknown correlations between internal diseases, personal medical data, and measurable skin properties will someday enable better diagnoses and health monitoring by employing different aspects of the invention.

Current instruments and techniques for obtaining generally noninvasive skin measurements are listed in Table 1. Some of these devices can already be miniaturized sufficiently enough to be incorporated into the wearable device of the invention. At present, other devices and techniques have not yet been miniaturized enough. Some laboratory tests require the further development of lab-on-a-chip technology. Unless otherwise noted, it is within the scope of this invention that all such instruments and techniques may be incorporated into wearable device 800 as technology progresses. Other devices and techniques useful for analyzing skin, sebum, perspiration and skin microbe samples may also be miniaturized in the future for both physical measurements and chemical analyses within the scope of this invention.

TABLE 1

| | MEASUREMENT | |
|---|---|---|
| INSTRUMENT/TECHNIQUE | TAKEN | COMMENTS |
| cutometer | elasticity | |
| Pulse oximeter | Blood oxygen | |
| corneometer | Epidermal hydration | |
| Towameter | Transepidermal | |
| | water loss | |
| ultrasound | subsurface properties | |
| spectrometers | detection of specific | |
| | chemicals | |
| colorimeters | skin color, flushing | |
| optical coherence tomography | 3D surface imaging | |
| traditional light microscopy | | * |
| Fourier transform infrared | | * |
| microscopy (FTIR) | | |
| scanning electron microscopy | | * |
| visible | | |
| microspectrophotometry | | |
| IR Thermometry | | |
| Moisture meter | | |
| Thermalcamera | | |
| Transluminescence probe | | |
| dermal torque | | |
| Torsional ballistometer | | |
| D-Squames | | |
| Sebutapes | | |
| cyanoacylate | | |
| Magnetic resonance | | * |
| Fringe Projection Topometry | | |
| Confocal microscopy | | * |
| Stereoimaging | | |
| Digital imaging | | |
| skin pH measuement | | |
| Capacitance Imaging | | |
| Dermal-Torquemeter | Skin mechanical | |
| | properties | |
| Video image analysis of | | |
| cutaneous replicae | | |
| Analysis of cutaneous | | |
| pictures under fringe | | |
| projection | | |
| SSA (skin surface analysis) | | |
| software | | |
| Reflectance Microscopy | | |
| Sonography | | |
| Fluoresence spectroscopy | | |
| Dynaskin 4D | | |
| High resolution photograph | | |
| analysis | | |
| Mexameter | | |
| Facescanner | | |
| microscopic scoring of skin | | * |
| stripping samples | | |
| Micro-depressionnary | | |
| network visualization | | |
| Visioscan | Skin surface | |
| | observation | |

* not likely amenable to wearable devices

For the purposes of this invention, the terms “skin measurement instruments” and “skin measurement techniques” refer only to instruments and techniques that measure the properties of skin and materials within the skin layer. These terms do not include devices or instruments applied to the skin surface to perform non-skin related measurements such as stethoscopes, EEGs or EKGs. However, such non-skin measurement devices and instruments may be integrated with the skin measurement devices, methods and systems of the invention.

Much of a user's medical data may be correlated with skin measurements to aid in improving the health of the user, as well as slowing the effects of aging. Additionally, advancement in the understanding of genetics and the development of genetic analysis tools will also increase the utility of the invention. The results of DNA sequencing, gene expression profiling, SNP analyses, and other techniques can be combined with other medical data and skin measurement data to monitor and improve skin health, to monitor and improve general health, and to monitor and detect specific diseases and physical conditions. The skin measurement data can be obtained via several aspects of the invention including medical centers 400, mobile devices 200, and wearable devices 800.

In one aspect the invention is a method for monitoring a specific medical condition of a user. The method comprises correlating the existence or severity of a selected medical condition with non-invasive skin measurements; selecting a plurality of non-invasive skin measurement instruments based on such correlating; incorporating the plurality of non-invasive skin measurement instruments into a device that is wearable by the user; and continuously taking skin measurements of the user by the device. The skin measurement instruments may be selected from the group consisting of thermometers, pH meters, moisture measuring instruments, light scattering instruments, and pulse oximeters. The skin measurement devices may be selected from the devices and techniques in Table 1.

In another aspect the invention is a wearable device for monitoring the health of an individual. The device comprises a skin contact area for providing continuous contact with a specific skin surface of the individual; a plurality of skin measurement instruments; and a transmitter for transmitting skin measurement data. The skin measurement instruments may be selected from the group consisting of thermometers, pH meters, moisture measuring instruments, light scattering instruments, and pulse oximeters. The skin measurement instruments may be selected from the devices and techniques in Table 1. The device may further comprise an air contact area having one or more air measurement devices; where the transmitter also transmits air measurement data. The air measurement devices may be selected from the group consisting of thermometers, ultraviolet light meters, hygrometers, and barometers.

In another aspect the invention is a system for monitoring the health of an individual. The system comprises a wearable device for taking measurements, the device having a skin contact area for providing continuous contact with a specific skin surface of the individual; a plurality of skin measurement instruments; and a transmitter for transmitting skin measurement data; a local unit having a first receiver for receiving skin measurement data; a transmitter for retransmitting the skin measurement data; and a second receiver; and a central computing unit. The skin measurement instruments may be selected from the group consisting of thermometers, pH meters, moisture measuring instruments such as Tewemeters®, light scattering instruments, pulse measuring devices, and pulse oximeters. The skin measurement instruments may be selected from the devices and techniques in Table 1. The wearable device may further comprise an air contact area and one or more air measurement devices, where the transmitter also transmits air measurement data. The air measurement devices may be selected from the group consisting of thermometers, ultraviolet light meters, hygrometers, and barometers.

While embodiments and applications of this invention have been shown and described, it would be apparent to those skilled in the art having the benefit of this disclosure that many more modifications than mentioned above are possible without departing from the inventive concepts herein. The invention, therefore, is not to be restricted except in the spirit of the appended claims.

What is claimed is:

1. A method for monitoring a specific medical condition of a user, the method comprising:
correlating the existence or severity of a selected medical condition with non-invasive skin measurements;
selecting a plurality of non-invasive skin measurement instruments based on such correlating;
incorporating the plurality of non-invasive skin measurement instruments into a device that is wearable by the user; and
continuously taking skin measurements of the user by the device.

2. The method of claim 1, where the skin measurement instruments are selected from the group consisting of thermometers, pH meters, moisture measuring instruments, light scattering instruments, and pulse oximeters.

3. A wearable device for monitoring the health of an individual comprising:
a skin contact area for providing continuous contact with a specific skin surface of the individual;
a plurality of skin measurement instruments; and
a transmitter for transmitting skin measurement data.

4. The device of claim 3, where the skin measurement instruments are selected from the group consisting of thermometers, pH meters, moisture measuring instruments, light scattering instruments, and pulse oximeters.

5. The device of claim 3, further comprising:
an air contact area; and
one or more air measurement devices;
where the transmitter also transmits air measurement data.

6. The device of claim 5, where the air measurement devices are selected from the group consisting of thermometers, ultraviolet light meters, hygrometers, and barometers.

7. A system for monitoring the health of an individual, the system comprising:
a wearable device for taking measurements, the device having a skin contact area for providing continuous contact with a specific skin surface of the individual; a plurality of skin measurement instruments; and a transmitter for transmitting skin measurement data;
a local unit having a first receiver for receiving skin measurement data; a transmitter for retransmitting the skin measurement data; and a second receiver; and
a central computing unit.

8. The system of claim 7, where the skin measurement instruments are selected from the group consisting of thermometers, pH meters, moisture measuring instruments such as Tewemeters®, light scattering instruments, pulse measuring devices, and pulse oximeters.

9. The system of claim 7, where the wearable device further comprises:
an air contact area; and
one or more air measurement devices;
and where the transmitter also transmits air measurement data.

10. The system of claim 9, where the air measurement devices are selected from the group consisting of thermometers, ultraviolet light meters, hygrometers, and barometers.
